(19) United States
(12) Patent Application Publication
(10) Pub. No.: US 2009/0269771 A1
(43) Pub. Date: Oct. 29, 2009

(54) METHOD OF SEQUENCING AND MAPPING TARGET NUCLEIC ACIDS

(75) Inventor: Benjamin G. SCHROEDER, San Mateo, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/430,005

(22) Filed: Apr. 24, 2009

Related U.S. Application Data
(60) Provisional application No. 61/047,651, filed on Apr. 24, 2008.

(51) Int. Cl. C12Q 1/68; C12P 19/34

(52) U.S. Cl. 435/6; 435/91.1

(57) ABSTRACT

The present teachings pertain to methods, compositions, reaction mixtures, and kits for mapping a low complexity sequence to a locus in a genome. In some embodiments, the low complexity sequence can be used to determine the methylation profile of a target nucleic acid. A strand-replacing reaction results in a product containing a first strand and a second strand, which can be connected together with a stem-loop adapter to form a single strand. A sequencing reaction can compare the two strands of the product, allowing the experimentalist to both map the sequence to a locus in a reference genome, as well as ascertain the methylation profile of the original target nucleic acid.

### FIELD

The present teachings pertain to methods, compositions, reaction mixtures, and kits for sequencing target nucleic acids.

### INTRODUCTION

Epigenomic changes to DNA provide another channel of information on which natural selection can act (see Goldberg et al., Cell, 128: 635-638). Increasing attention is being paid to methylation of bases in nucleic acids as one important epigenomic change. Methylation of bases can take different forms. For example, methylation of DNA by the DNA adenine methyltransferase (Dam) provides an epigenetic signal that influences and regulates numerous physiological processes in the bacterial cell including chromosome replication, mismatch repair, transposition, and transcription (see Heusipp et al., Int J Med. Microbiol. 2007 February; 297(1):1-7, Epub 2006 Nov. 27 for a review). Also, methylation of cytosine in mammals at CpG dinucleotides correlates with transcriptional repression, and plays a crucial role in gene regulation and chromatin organization during embryogenesis and gametogenesis (Goll and Bestor (2006) Annu. Rev. Biochem. 74, 481-514).

One method of measuring the presence of cytosine methylation takes advantage of the ability of the converting agent bisulfite to convert non-methylated cytosines to uracil (See Boyd et al., Anal Biochem. 2004 Mar. 15; 326(2), 278-80, Anal Biochem. 2006 Jul. 15; 354(2):266-73. Epub 2006 May 6, and Nucleosides Nucleotides Nucleic Acids, 2007; 26(6-7):629-34. After such conversion, a sequence amplified in a PCR bears thymine at those residues that were originally unmethylated cytosine. However, methylated cytosines are protected from such bisulfite treatment. Accordingly, the presence of a thymine at a location known to normally contain cytosine reflects that the original cytosine was unmethylated. Conversely, the presence of a cytosine at a location known to normally contain cytosine reflects that the original cytosine was methylated.

Following bisulfite conversion, and PCR amplification, sequences containing a large number of unmethylated cytosines will have a low complexity, since the non-methylated cytosines will have been converted to thymine, and the resulting sequence will be dominated by only three bases (A, G, and T). Such low complexity sequences can be difficult to map to a region (locus) of the genome. That is, when a low complexity nucleic acid is sequenced, it can be difficult to know what part of the genome the sequence comes from. Such a problem is particularly acute in various sequencing approaches that employ short read-lengths.

### SUMMARY

In some embodiments, the present teachings provide a method of determining the methylation profile of a target nucleic acid comprising, ligating a first adapter to an extendable 3′ end of the target nucleic acid, wherein the first adapter is a stem-loop molecule comprising an extendable 3′ end and a phosphorylated 5′ end, wherein the target nucleic acid comprises a native first strand and a complementary second strand, and wherein a nick is between the 3′ extendable end of the first adapter and the second strand of the target nucleic acid; extending the 3′ end of the stem-loop adapter with dATP, dGTP, dTTP, 5-methyl-dCTP to form a fully methylated strand, wherein the fully methylated strand is complementary to the first native strand; providing a second adapter, wherein the second adapter comprises a first strand and a second strand, wherein the first strand comprises a first primer portion, and an extendable 3′ end, and the second strand comprises a second primer portion and a phosphorylated 5′ end; ligating the fully methylated second strand to the phosphorylated 5′ end of the second adapter and ligating the first native strand of the target nucleic acid to the extendable 3′ end of the second adapter, to form a dual-adapter ligation product; converting non-methylated cytosine in the first native strand of the dual-adapter ligation product to uracil to form a converted native strand in a converted dual-adapter ligation product; immobilizing the converted dual-adapter ligation product on a solid support; hybridizing a primer to the second primer portion of the converted dual-adapter ligation product; sequencing the converted dual-adapter ligation product; and, comparing the identity of the cytosine positions in the fully-methylated second strand with the identity of the cytosine positions in the converted strand to determine the methylation profile of the target nucleic acid.

In some embodiments, the present teachings provide a method of determining the methylation profile of a target nucleic acid comprising; ligating a first adapter to an extendable 3′ end of the target nucleic acid, wherein the first adapter is a stem-loop molecule comprising an extendable 3′ end and a phosphorylated 5′ end, wherein the target nucleic acid comprises a native first strand and a complementary second strand, and wherein a nick is between the 3′ extendable end of the first adapter and the second strand of the target nucleic acid; extending the 3′ end of the stem-loop adapter with dATP, dGTP, dTTP, 5-methyl-dCTP to form a fully methylated strand, wherein the fully methylated strand is complementary to the first native strand; providing a second adapter, wherein the second adapter comprises a first strand and a second strand, wherein the first strand comprises a first primer portion, and an extendable 3′ end, and the second strand comprises a second primer portion and a phosphorylated 5′ end; ligating the fully methylated second strand to the phosphorylated 5′ end of the second adapter and ligating the first native strand of the target nucleic acid to the extendable 3′ end of the second adapter, to form a dual-adapter ligation product; converting non-methylated cytosine in the first native strand of the dual-adapter ligation product to uracil to form a converted native strand in a converted dual-adapter ligation product; immobilizing the converted dual-adapter ligation product on a solid support; hybridizing a primer to the second primer portion of the converted dual-adapter ligation product; sequencing the converted dual-adapter ligation product; and, comparing the identity of the cytosine positions in the fully-methylated second strand with the identity of the cytosine positions in the converted strand to determine the methylation profile of the target nucleic acid.

In some embodiments, the present teachings provide a method of forming a single-stranded dual-adapter ligation product comprising; forming an adapter-ligated single-stranded target nucleic acid; hybridizing a primer to the adapter of the adapter-ligated single-stranded target nucleic acid; extending the primer in the presence of 5-methyl dCTP to form a double-stranded product comprising a fully methylated strand; and, ligating a stem-loop adapter to the double-stranded product to form a single-stranded dual adapter ligation product.

More generally, in some embodiments the present teachings provide a method of mapping a low complexity sequence to a locus of a genome comprising; generating a strand replacement product comprising a high complexity strand and a low complexity strand; sequencing the high complexity strand; and, comparing the sequence of the high complexity strand to the genome in order to map the low complexity strand to a locus of the genome.

Kits, compositions, and reactions mixtures are also provided.

### BRIEF DESCRIPTION OF THE DRAWINGS

FIG. 1 shows one illustrative embodiment according to the present teachings.

FIG. 2 shows one illustrative embodiment according to the present teachings.

FIG. 3 shows one illustrative embodiment according to the present teachings.

FIG. 4 shows one illustrative embodiment according to the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### SOME DEFINITIONS

As used herein, term “dephosphorylated 5′ end” refers to a nucleic acid in which the 5′ end lacks phosphate groups, and is generally unable to ligate to an extendable 3′ end as result of the absence of the phosphate groups.

As used herein, the term “target nucleic acid” refers generally to a nucleic acid under inquiry. In some embodiments, the target nucleic acid is that whose methylation profile is to be determined. For convenience, target nucleic acids are referred to as containing a “first strand” and a complementary “second strand”.

As used herein, the term “fully methylated strand” refers to the strand that results from the strand replacement reaction, and for example can incorporate methylated cytosines.

As used herein, the term “first adapter” refers to a double-stranded nucleic acid which contains a 5′ phosphorylated end and a 3′ extendable end. In some embodiments, the first adapter can be a stem-loop adapter. In some embodiments, the first adapter can be a blunt-ended double-stranded adapter. In some embodiments, the first adapter can be a sticky-ended double-stranded adapter.

As used herein, the term “double-stranded stem of the first adapter” refers to a double-stranded portion of the first adapter. In some embodiments, non-methylated cytosines can be included in the double-stranded stem of the first adapter that can be converted by the converting agent. As a result, following conversion with bisulfite for example, the first strand and the second strand of the double-stranded stem of the first adapter are no longer complementary, thus increasing the likelihood that the converted dual-adapter ligation product will be single-stranded.

As used herein, the term “stem-loop adapter” refers to a molecule comprising a double-stranded stem with a single-stranded loop region disposed between the two strands that comprise the double-stranded stem. The stem-loop adapter further comprises a 5′ phosphorylated end and a 3′ extendable end.

As used herein, the term “extendable 3′ end” refers to the ability of the 3′ end of a molecule, such as a stem-loop adapter for example, to be extended by a polymerase thru the addition of nucleotides, thus elongating the molecule. Generally, the 3′ end can contain a hydroxyl group at the 3′ position of the sugar of the nucleotide.

As used herein, the term “phosphorylated 5′ end” refers to the phosphate that occurs at the 5′ end of a nucleic acid, and which generally forms the substrate for a ligation reaction which can join such a 5′ phosphate group with a 3′ OH group. In some embodiments, the phosphorylated 5′ end results from an experimentally performed phosphorylation reaction, for example a phosphorylation reaction using a kinase. Removal of such a phosphorylated 5′ end is referred to herein as “de-phosphorylation”, which can be achieved for example by the use of a phosphatase. De-phosphorylation results in a “de-phosphorylated 5′ end”.

As used herein, the term “converting” refers to the use of certain agents, for example bisulfite, which can preferentially alter nucleotide residues, thus forming a low complexity strand. For example, non-methylated cytosines can be converted by bisulfite to a different residue, uracil. Accordingly, the term “converting agent” refers to one of such agents.

As used herein, the term “converted native strand” refers to the result of a converting reaction, for example converting with bisulfite, where for example the non-methylated cytosines of the native strand of a target nucleic acid are converted to uracils. In some embodiments, the present teachings will refer to a “non-converted native strand.” Such a non-converted native strand is merely a native strand of a target nucleic acid which has not undergone a conversion reaction.

As used herein, the term “ligating” refers to any chemical, enzymatic, or other means of attaching the end of one nucleic acid to another. For example, the covalent attachment of the 5′ phosphate of a stem-loop adapter to the extendable 3′ end of a target nucleic acid by the use of a ligase enzyme is one example of ligating.

As used herein, “sequencing” and sequencing reagents refer to methods and compositions used to determine the sequence of nucleotides in a target nucleic acids. For example, polymerase-mediated sequencing such as a Sanger di-deoxy chain terminators, and reversible terminators. Another example is various ligation-mediated sequencing approaches that employ ligation probes, for example as taught in Published US Patent Application US20080003571A1.

As used herein, the term “methylation profile” refers to the particular pattern of methylated residues in a target nucleic acid. Such methylation profiles of the present teachings can be ascertained by comparing the sequence of the fully methylated strand with the converted strand. Those nucleotide positions in the fully methylated strand that are determined to be C (and thus G in a sequencing reaction), while the corresponding nucleotide position in the converted strand are U (and T following a PCR, and thus A in a sequencing reaction), can be inferred to be a cytosine position that was methylated in the original strand. Comparing a number of such G/A differences in the fully methylated strand with the converted strand allows one to determine a methylation profile.

As used herein, the term “5-methyl-dCTP” refers to a methylated version of cytosine of the chemical formula 5-methyl-2′-deoxycytidine-5′-triphosphate. Generally, 5-methyl-dCTP's can be included in the strand replacement reaction, thus resulting in the formation of a fully methylated strand.

As used herein, the term “dual-adapter ligation product” refers to a strand replacement product, which has undergone a strand replacement reaction to incorporate an altered residue, such as for example 5-methyl-dCTP, and to which a second adapter has been ligated.

As used herein, the term “converted dual-adapter ligation product” refers to a dual-adapter ligation product that has been treated with a converting agent such as bisulfite, thus for example converting the unmethylated cytosine of the native strand to uracil.

As used herein, the term “strand replacement product” refers to the result of a strand replacement reaction such as nick translation or any other primer extension reaction. The strand replacement product can contain a native first strand, and a fully methylated strand that results from primer extension.

As used herein, the term “shortened strand replacement product” refers to a strand replacement product whose length has been reduced, for example by undergoing a cleavage reaction with a distal cutting restriction enzyme.

As used herein, the term “affinity moiety” refers to any of a variety of compounds that can be incorporated into a nucleic acid and which can selectively bind an “affinity moiety binding agent”, thus allowing for immobilization of the entity bearing the affinity moiety. Biotin is an example of an affinity moiety; streptavidin is an example of a corresponding affinity moiety binding agent.

As used herein, the term “distal-cutting restriction enzyme” refers to any of a variety of restriction enzymes that recognize a particular nucleic acid sequence (a recognition site), and cut a distance away from that recognition site. Type IIs restriction enzymes are one example of a class of distal-cutting restriction enzymes.

As used herein, the term “primer” refers generally to a sequence of nucleotides that can initiate a subsequent extension of that sequence of nucleotides, and which is generally complementary to an underlying nucleic acid. For example, a primer can contain an extendable 3′ end in the form of a hydroxyl group at the 3′ position of the sugar of the 3′-most base, thus allowing a polymerase to extend the primer with free nucleotides.

As used herein, the term “enzyme-mediated extension reaction” refers to both polymerase and/or ligase-mediated reactions in which elongation of an oligonucleotide occurs.

As used herein, the term “strand-replacing polymerase” refers to any of a variety of polymerases that can effectuate the generation of a second strand, for example a fully methylated strand. Example of strand-replacing polymerases are strand-displacing polymerase such as Bst and Phi29. Another example of a strand-replacing polymerase is an exonuclease-containing polymerase such as E. Coli DNA polymerase I, which can be used in a nick translation reaction. In some embodiments, a strand-replacing polymerase is any of a variety of polymerases that merely function to polymerize nucleotide addition into a complementary strand, the earlier strand having been removed by denaturation.

As used herein, the term “strand-displacing polymerase” refers to a polymerase that has the property of extending through pre-existing nucleotides in a strand, thus forming a new strand in its place. Bst and Phi29 are two examples of strand-displacing polymerases.

As used herein, the term “cytosine positions” refers to the place in a sequence where a cytosine residue occurs. For example, in the sequence 5′CTACG3′, there are two cytosines. The first cytosine is in position one. The second cytosine is in position four. A given cytosine position can have an identity as being either methylated or unmethylated. Correspondingly, “adenine positions” refers to a place in a sequence where an adenine occurs.

As used herein, the term “single nucleic acid strand” refers generally to a single chain molecule of repeating nucleotides, comprising a 3′ end and a 5′ end. A dual-adapter ligation product is one example of a single nucleic acid strand. Another example of a single nucleic acid strand is a converted dual-adapter ligation product. Another example of a single nucleic acid strand is a strand replacement product. Another example of a single nucleic acid strand is a shortened strand replacement product.

As used herein, the term “nick translation” refers to a polymerase-mediated reaction in which a pre-existing strand is displaced and replaced by the 5′ to 3′ exonuclease activity of a polymerase, to result in a novel strand. E. Coli DNA polymerase I is one example of such a polymerase. The nick translating reactions performed according to the present teachings can contain a 5-methyl-dCTP, such that the resulting product, a fully methylated strand, contains methylated cytosine at the cytosine positions.

As used herein, the term “low complexity sequence” refers to a sequence that does not contain 25 percent A, 25 percent G, 25 percent C, and 25 percent T, but rather contains at least 80 percent, at least 85 percent, at least 90 percent, at least 95 percent, or at least 99 percent of three of the four bases.

As used herein, the term “high complexity sequence” refers to a sequence that contains 25 percent A, 25 percent G, 25 percent C, and 25 percent T, or no less than 15 percent of any one of the four bases, no less than 10 percent of any one of the four bases, or no less than 5 percent of any one of the four bases.

Other terms as used herein will harbor meaning based on the context, and can be further understood in light of the understanding of one of skill in the art of molecular biology. Illustrative teachings describing the state of the art can be found, for example, in Sambrook et al., Molecular Cloning, 3rd Edition. It will be appreciated that the primers and nucleotides employed in the present teachings can include any of a variety of known analogs, including LNA, phosphorothiolate compounds, as well as any of a variety of known analogs of the sugar, base, and/or phosphate backbone.

### DETAILED DESCRIPTION OF THE DRAWINGS

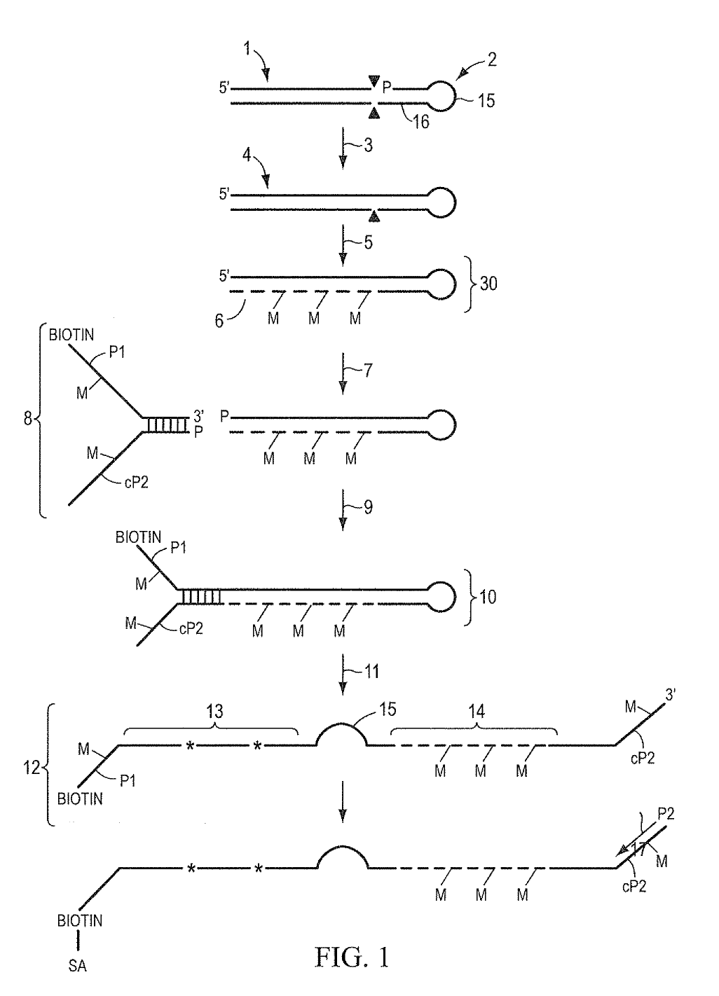

One embodiment of the present teachings is shown in FIG. 1. Here, a double stranded target nucleic acid (1) is shown containing a first strand (top horizontal line) and a second strand (bottom horizontal line). A first adapter (2) is also shown. The first adapter contains a phosphate group (P) at its 5′ end, referred to herein as a “phosphorylated 5′ end.” The first adapter also contains a double-stranded stem (16), and a loop (15). The target polynucleotide is shown with dephosphorylated 5′ ends (note the absence of a (P) on the left end of the first strand, and the absence of a (P) on the right end of the second strand). The absence of phosphate groups on the 5′ end of the first strand of the target nucleic acid prevents target polynucleotides from ligating to one another, thus minimizing the occurrence of an unwanted side reaction. The absence of phosphate groups on the 5′ end of the second strand of the target nucleic acid prevents the first adapter from ligating to this end, thus leaving a nick (note triangles) following treatment with a ligase. As shown in (3), the 5′ phosphate group of the first adapter can be ligated to the extendable 3′ end of the first strand in a ligation reaction to form a first ligation product (4).

A nick (note the triangle between the second strand of the target nucleic acid and the 3′ extendable end of the adapter) between the 5′ dephosphorylated end of the second strand, and the extendable 3′ end of the adapter, can be taken advantage of by performing a strand replacement reaction, such as nick translation. Thus, following the ligation reaction, a strand replacement reaction (5) can be performed to form a strand replacement product (30). In such a strand replacement reaction, a polymerase possessing 5′ to 3′ exonuclease activity can be used, along with dTTP, dGTP, dATP, and 5-methyl-dCTP. The result of this strand replacement reaction is a strand replacement product comprising a fully methylated strand (6, note the M's indicating methylated cytosine incorporation) and a native strand. Accordingly, all the cytosines in the fully methylated strand are now methylated. This is contrasted with the cytosines in the native (top) strand, which remain in their normal state, some being methylated and others not.

Following the strand replacement reaction, a phosphorylation reaction (7) can be performed, which results in the addition of a phosphate group to the 5′ end of the native strand (indicated by the presence of the P on the left side of the top strand). A second adapter (8) can then be provided. The second adapter can contain a first strand comprising a first primer portion (P1), an affinity moiety (here, Biotin), and an extendable 3′ end (3′), and a second strand containing a second primer portion (cP2) and a phosphorylated 5′ end (P). Regions of complementarity between the first strand of the second adapter and the second strand of the second adapter form a double-stranded stem (note vertical lines indicating hydrogen-bonding between complementary base-pairs). Additionally, both strands of the second adapter can contain methylated cytosines (shown as M). The presence of methylated cytosines in the second adapter can serve the function of protecting these cytosine residues from the subsequent conversion treatment.

Ligating (9) the second adapter to the strand replacement product results in a dual-adapter ligation product (10). This dual-adapter ligation product can then be treated with a converting agent (11) such as bisulfite. Bisulfite converts the un-methylated cytosines in the first strand into uracils (shown as two *'s), to form a converted strand (13) in a converted dual-adapter ligation product (12). The methylated cytosines in the fully methylated strand (14) are resistant to treatment with bisulfite, and remain as methylated cytosines. As a result of the bisulfite treatment and resulting change in unmethylated cytosine to uracil, the two strands of the converted dual-adapter ligation product are no longer completely complementary, thus facilitating their disassociation to form a single nucleic acid strand. The single nucleic acid strand comprises the fully methylated strand (14) and the converted native strand (13). Disposed between the fully methylated strand (14) and the converted strand (13) is remaining loop sequence from the original first adapter (2), shown for orientation here as a hump (15). Also disposed between the fully methylated strand (14) and the converted native strand (13) can be the converted first adapter, which can contain the double-stranded stem of the first adapter. Such double-stranded stem can now be non-complementary as a result of conversion of certain of its non-methylated cytosine by the bisulfite. The converted dual-adapter ligation product (12) can be immobilized, for example by taking advantage of an affinity moiety binder such as streptavidin (SA) and its affinity for the biotin incorporated into the converted dual-adapter ligation product. Such immobilization can allow for the separation of the desired reaction products from unincorporated reaction products, thus improving the efficiency of downstream reactions.

Comparing the sequence of the converted native strand (13) with the sequence of the fully methylated strand (14) allows for the determination of the methylation profile of the original double-stranded target nucleic acid (1). Such a comparison can be achieved by sequencing. For example, a primer (17, P2) can be hybridized to its complementary primer portion (cP2) in the converted dual-adapter ligation product, and any of a variety of sequencing approaches performed, such as Sanger-di-deoxy sequencing, ligation-mediated sequencing, polymerase-mediated sequencing with reversible terminators, etc.

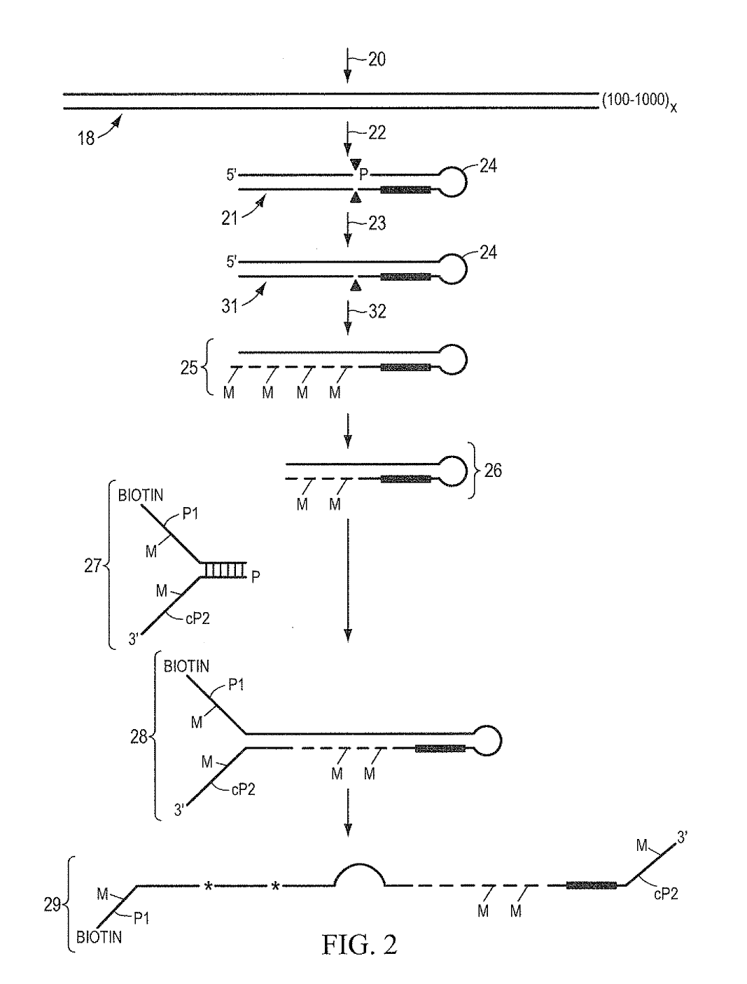

In some embodiments, the experimentalist may wish to start with a larger double stranded target nucleic acid. Further, the experimentalist may wish to use a sequencing approach to determine the methylation profile that employs short-fragment reads. In one embodiment of the present teachings, a larger target nucleic acid is used, and subsequent manipulations allow for its decrease in size, thus making the fragment compatible with short-fragment sequencing approaches. Such an embodiment is depicted in FIG. 2.

In FIG. 2, a sample can be prepared ((20) to provide a target nucleic acid (18). Such a target can be any size, for example on the order of a few hundred to several thousand nucleotides in length (100-1000)×. The length of such target nucleic acids can be shortened by any of a variety of procedures (22), such as shearing, enzymatic digestion and various procedures, including the commercially available HYDROSHEAR™ system. Such procedures can be optimized to ensure optimal representation of various regions of the genome in the eventual sample to be sequenced. After such a process (22), a collection of shorter fragments results, one of which is shown as (21). Such shorter fragments can be blunt-ended, using conventional polymerase-mediated blunting strategies. Additionally, such shorter fragments can be dephosphorylated, thus forming dephosphorylated 5′ ends. The absence of a phosphate group on the 5′ end of the second strand of the fragment prevents the first adapter (24) from ligating to this end, thus leaving a nick (note the triangle, representing the gap between the 5′ end of the second strand and the extendable 3′ end of the adapter following ligation). However, the extendable 3′ end of the first strand can ligate to the phosphorylated 5′ end of the adapter to form a first ligation product (31). The nick between the dephosphorylated 5′ end of the second strand, and the extendable 3′ end of the adapter, can be taken advantage of by performing a strand replacement reaction, such as nick translation.

Following the strand replacement reaction (32), the resulting strand replacement product (25) can be treated with a type IIs restriction enzyme. A type IIs restriction enzyme sequence present in the adapter (rectangle) can be recognized by the enzyme, and the enzyme cuts a distance away from the recognition site. Given the cut-site's location in the fragment, a further shortening of the size of the fragment occurs, resulting in a shortened strand replacement product (26). The shortened strand replacement product can be blunt ended and phosphorylated as necessary, and a second adapter (27) ligated to it to form a dual-adapter ligation product (28), which can be manipulated in any fashion, for example by being converted into a converted dual-adapter ligation product (29), and further manipulated as discussed in FIG. 1.

Thus, in some embodiments the present teachings provide a method of forming a single nucleic acid strand that contains a sequence comprising a first native strand and a fully methylated strand, the method comprising; ligating a first adapter to a 3′ end of a target nucleic acid to form a first ligation product, wherein the first ligation product comprises a nick between the 3′ end of the adapter and the target nucleic acid, wherein the first adapter is a stem-loop adapter comprising an extendable 3′ end and a phosphorylated 5′ end, and wherein the first adapter further comprises a distal-cutting restriction enzyme recognition site, wherein the target nucleic acid comprises a first native strand and a complementary second strand, wherein the target nucleic acid comprises a dephosphorylated 5′ end; extending the extendable 3′ end of the stem-loop adapter with dATP, dGTP, dTTP, 5-methyl-dCTP to form a strand replacement product, wherein the strand replacement product comprises a fully methylated strand, wherein the fully methylated strand is complementary to the first strand; and, cleaving the strand replacement products with a distal-cutting restriction enzyme to form a single nucleic acid strand that contains the first native strand and a fully methylated strand. In some embodiments the extending occurs after the cleaving. In some embodiments, the extending occurs before the cleaving. In some embodiments, the single nucleic acid strand is seventy-five to one-hundred and seventy-five nucleotides long.

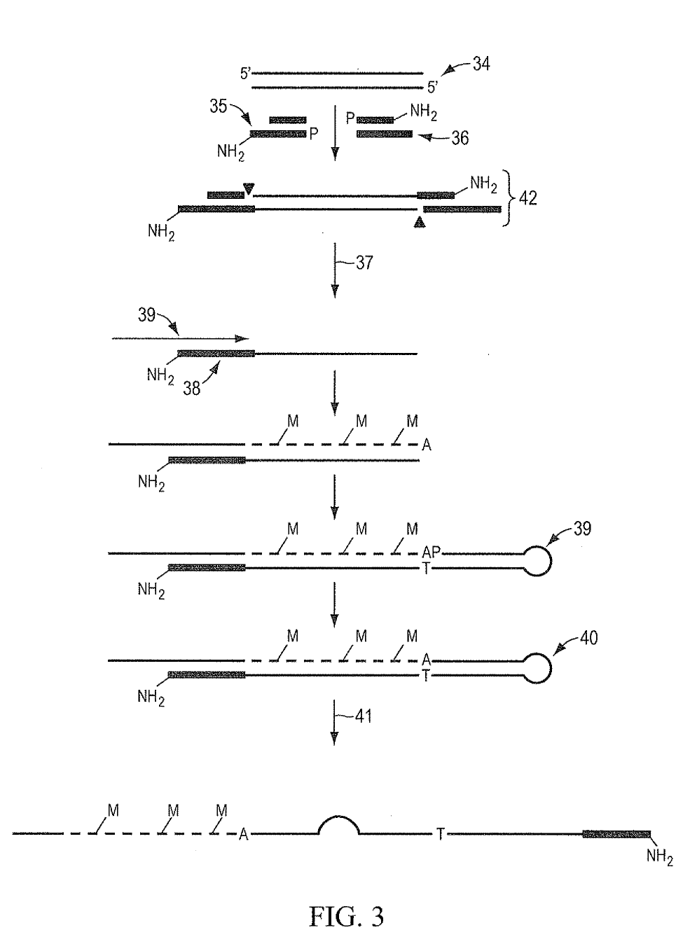

In some embodiments, the first step of the method need not employ ligation of a stem-loop adapter to a target nucleic acid, but rather can employ an enzyme-mediated extension reaction of a single-stranded primer, and the stem-loop adapter can thereafter be ligated to the resulting newly synthesized strand. Such an enzyme-mediated extension reaction can be considered a kind of strand replacement reaction. An embodiment is depicted in FIG. 3 were a dephosphorylated double stranded target nucleic acid (34) can be ligated to linear double stranded adapters (35 and 36). The resulting ligation product (42) contains nicks (note triangles) as a result of the absence of phosphate groups on the 5′ ends of the double stranded target nucleic acid. After a clean up and heat treating (37) to make a single nucleic acid strand (38), a single-stranded primer (39) can be hybridized at or near the 3′ end of the single nucleic acid strand and an enzyme-mediated extension reaction can be performed with a mix of dATP, dTTP, dGTP, and 5-methyl dCTP, to form a fully methylated strand (note M's, indicating incorporation of 5-methyl dCTP). The 3′ ends of the adapters can contain a blocking moiety, such as an amine (NH2) group, thereby preventing unwanted extension of the adapter by the polymerase. The extension reaction can employ a polymerase that leaves a template-independent A (note the A) at the 3′ end of the newly synthesized fully methylated strand. (In some embodiments, a template-independent A need not be introduced, and the subsequent adapter ligation reaction can be blunt-ended). The depicted A overhang can then form a complementary base-pairing interaction with the T of a stem-loop adapter (39). As a result of a phosphorylated 5′ end (note the P) on the stem-loop adapter, the A overhang can ligate to the stem-loop adapter to form a dual-adapter ligation product (40). The resulting dual-adapter ligation product contains a fully methylated strand (top strand) and a native strand (bottom strand). Following a treatment with heat (41), a single-stranded dual-adapter ligation product results, which can be treated with a conversion agent such as bisulfite, and then amplified and sequenced. Comparing the identity of the base (C or T) of the cytosine positions between the fully methylated strand and the native strand allows the experimentalist to determine the methylation signature of the original target nucleic acid.

In some such embodiments, the single-stranded primer can comprise methylated cytosines, and accordingly will be protected by treatment with a conversion agent such as bisulfite. In some such embodiments, the single-stranded primer need not comprise methylated cytosines, and can contain normal unmethylated cytosines, and accordingly will be susceptible to conversion by treatment with a conversion agent such as bisulfite.

Thus, in some embodiments the present teachings provide a method of forming a single-stranded dual-adapter ligation product comprising forming an adapter-ligated single-stranded target nucleic acid; hybridizing a primer to the adapter of the adapter-ligated single-stranded target nucleic acid; extending the primer in the presence of 5-methyl dCTP to form a double-stranded product comprising a fully methylated strand; and, ligating a stem-loop adapter to the double-stranded product to form a single-stranded dual adapter ligation product. In some embodiments, the dual-adapter ligation product is treated with a converting reagent, and methylation status ascertained according to the present teachings.

Non-Complementarity Between Strands of the First Adapter in the Converted Dual-Adapter Ligation Product can Increase Likelihood of Single-Strandedness

As shown and described in FIG. 1, disposed between the fully methylated strand (14) and the converted strand (13) is the converted first adapter, containing the double-stranded stem of the first adapter. This double-stranded stem can now be non-complementary as a result of conversion of certain of its non-methylated cytosines by the bisulfite converting treatment. Thus, in some embodiments of the present teachings, non-methylated cytosines can be embedded into the stem of the first adapter, thus allowing for their conversion. This conversion increases the mismatches between the first strand and the second strand of the double-stranded stem of the first adapter, thus increasing the likelihood that the converted dual-adapter ligation product exists in single-stranded form. In some embodiments, at least two non-methylated cytosines are included in one strand of the stem of the first adapter. In some embodiments, at least three, at least four, at least five, at least six, at least seven, at least eight, at least nine, at least ten, at least eleven, or at least twelve non-methylated cytosines are included in one strand of the stem of the first adapter. In some embodiments, two to eight non-methylated cytosines are included in one strand of the double-stranded stem of the first adapter. In some embodiments, three to seven non-methylated cytosines are included in one strand of the stem of the first adapter. In some embodiments, four to six non-methylated cytosines are included in one strand of the stem of the first adapter.

### Illustrative Mapping of a Converted Strand and a Fully-Methylated Second Strand

Following bisulfite conversion, and PCR amplification, sequences containing a large number of unmethylated cytosines will have a low complexity, since the non-methylated cytosines will have been converted to thymine, and thus this low complexity sequence will be dominated by three bases, instead of four. Generating meaningful data from conventional sequencing of bisulfite-converted DNA is plagued by this low sequence complexity of the resulting sequence data. This lower complexity sequence is more difficult to map to a region of a known genomic locus than a sequence of the same length that contains all four bases, A, T, G, and C. According to the present teachings, sequencing the converted dual-adapter ligation product can facilitate mapping the resulting information to regions of a known genome. Thus, the converted dual-adapter ligation product provided by the present teachings provides a simplified way of mapping a low complexity sequence to a region of a known genome. The fully methylated strand maintains its complexity; it has all four bases. The fully methylated strand can thus be used to determine the region of the known genome to which the converted native strand maps. That is, the relatively low complexity converted native strand can take advantage of the mapping information provided by the fully methylated strand. Further, by comparing the sequence information collected from the low complexity converted native strand, to the sequence information collected from the high complexity fully methylated strand, the experimentalist can determine the methylation profile of the original target nucleic acid. Such a methylation profile follows from comparing those T's in the converted native strand that are present in the same cytosine position as the corresponding cytosines in the fully methylated strand. These two pieces of sequence information arise from a single source; the single strand that is sequenced.

Thus, after forming a converted dual-adapter ligation product, the fully methylated strand can be sequenced. This sequence can be compared to a known genomic consensus sequence to determine where in the genome the sequence maps. The sequence of the converted native strand can then be compared to the sequence of the fully methylated strand. Differences in the cytosine position between the sequence collected for the converted strand, compared to the sequence collected for the fully methylated strand, indicates where in the original target nucleic acid cytosines were methylated. As will be appreciated, any ordering of such steps can be performed according to the present teachings.

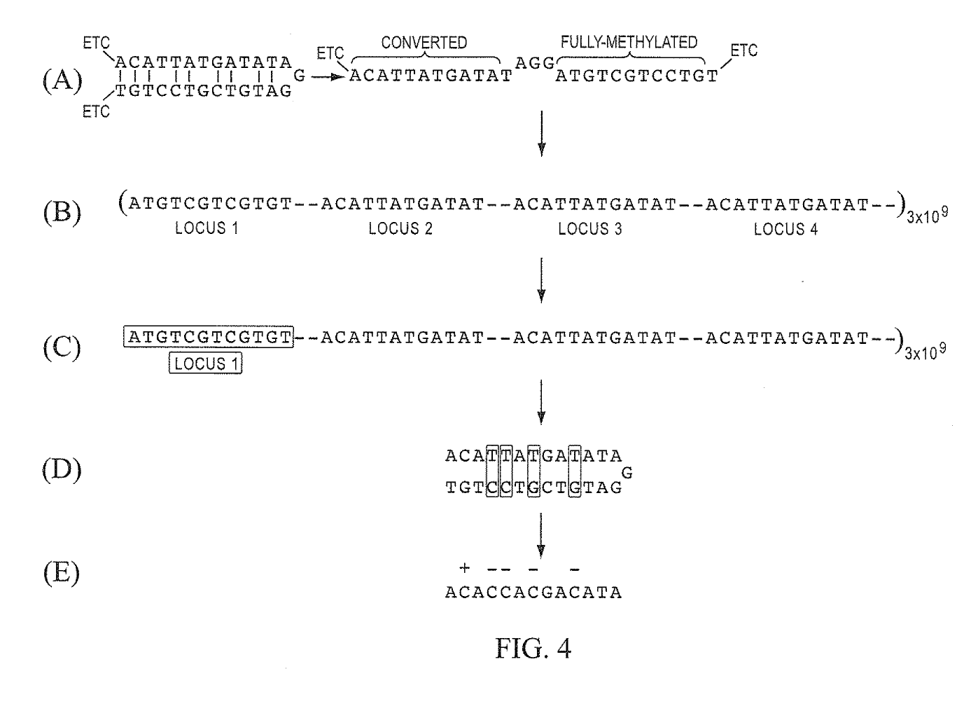

FIG. 4 illustrates such a mapping procedure. Here, a strand replacement product is shown in (A). Note the non-complementary T-C pairings, indicative of conversion of non-methylated cytosines to U, and thereafter to C in a PCR. A full length single-stranded representation of the relevant portions of a converted dual-adapter ligation product is shown to the right in (A). Note that the converted native strand contains only a single C. Thus, the converted native strand is of low complexity; it is dominated by just three bases. Contrast this with the fully methylated strand, which contains all four bases in somewhat similar proportions.

FIG. 4 (B) depicts the human genome, a sequence roughly 3 billion bases in length (3×10⁹). Such a long sequence can be expected to have numerous occurrences of any given low complexity sequence. To take an extreme example, the sequence AAA appears numerous times in the human genome. When a sequencing reaction produces AAA, it is impossible to know to which of the numerous such loci in the genome such a sequence maps. In (B) a first locus is shown (Locus 1), which contains the sequence of the fully methylated strand. Locus 2, Locus 3, and Locus 4 represent various loci throughout the genome that have the same sequence as the converted native strand. Comparing the sequence of the converted native strand to the full-length genome sequence thus raises the question: to which locus does the converted native strand map? The converted native strand could map to Locus 2, or to Locus 3, or to Locus 4. Further, simply considering the sequence of the converted strand says nothing as to methylation status. Any of the T's in the converted strand could a bona-fide T in the target nucleic, or, on the other hand could represent a non-methylated C that got converted to U, and further to T in a PCR.

Contrast this to the fully methylated strand. This strand has four bases, and is thus of higher complexity. There is only one locus in the genome to which this sequence maps: Locus 1. This is depicted in FIG. 4 (C). Thus, comparing the sequence of the fully methylated strand to the referent genome allows for the determination of where in the genome the sequence derives. Here, the experimentalist knows that the sequence of interest maps to locus 1.

Next, the experimentalist can compare the sequence of the converted native strand to the sequence of the fully methylated strand. As indicated in FIG. 4 (D), those areas where a T is in a cytosine position represents cytosines that were originally unmethylated. Finally, in FIG. 4(E) a sequence is shown that represents the methylation profile of the original target nucleic acid. As shown, only one of the cytosines in the original target nucleic was methylated (note single plus). Four cytosines in the original target nucleic acid were unmethylated (note the four minuses).

While the examples use methylation as the application area for illustrating one embodiment of the present teachings, the present teachings more generally provide an improved method of mapping a low complexity sequence to a locus of a genome. In some embodiments, the method comprising generating a strand replacement product comprising a high complexity strand and a low complexity strand; sequencing the high complexity strand; and, comparing the sequence of the high complexity strand to the genome in order to map the low complexity strand to a locus of the genome. In some embodiments, the high complexity strand is a fully-methylated first strand and the low complexity strand is a converted strand. In some embodiments, the fully methylated strand comprises cytosines that are methylated, and the strand-replacing reaction comprises 5-methyl-dCTP. In some embodiments, the fully methylated strand comprises adenines that are methylated, and the strand replacing reaction comprises methylated adenines.

### Compositions and Reaction Mixtures

The present teachings further provide novel reaction mixtures. For example, in some embodiments, the present teachings provide a reaction mixture comprising; (a) an adapter ligated to a first strand of a target nucleic acid, wherein the target nucleic acid comprises a first strand and a second strand, wherein the adapter is a stem-loop adapter comprising an extendable 3′ end, and, wherein a nick exists between the extendable 3′ end of the stem-loop adapter and the second strand of the target nucleic acid; (b) a strand-replacing polymerase; (c) 5-methyl-dCTP; and, (d) at least one of dATP, dTTP, dGTP.

In some embodiments, the present teachings provide a reaction mixture comprising; (a) a dual-adapter ligation product; and, (b) bisulfite.

In some embodiments, the present teachings provide a reaction mixture comprising a strand replacement product comprising a fully methylated strand; and, bisulfite.

In some embodiments, the present teachings provide for novel compositions. For example, in some embodiments, the present teachings provide a strand replacement product, wherein the strand replacement product comprises a high complexity second strand and a low complexity first strand. In some embodiments, the high complexity second strand comprises 5-methyl-dCTP.

### Kits

The present teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In some embodiments, the present teachings provide a kit for determining the methylation profile of a target nucleic acid comprising; (a) a first adapter, wherein the first adapter is a stem-loop adapter, and wherein the stem-loop adapter comprises a phosphorylated 5′ end and an extendable 3′ end; (b) a second adapter, wherein the second adapter comprises a phosphorylated 5′ end; (c) a strand-replacing polymerase; (d) a converting agent; (e) a kinase; (f) 5-methyl-dCTP; and, (g) at least one of dATP, dTTP, dGTP. In some embodiments, the kits of the present teachings can further comprise at least one of (h) a distal-cutting restriction enzyme, or (i) sequencing reagents. In some embodiments, the sequencing reagents comprise at least one polymerase, or at least one ligase. In some embodiments, the kits comprise at least one converting agent, such as for example bisulfite.

In some embodiments, the present teachings provide a kit comprising a primer, 5-methyl-dCTP, polymerase, dAGT, and bisulfite. In some embodiments, the kit comprises a strand displacing polymerase. In some embodiments, the kit comprises a stem-loop adapter.

### Example 1

One microgram of genomic DNA is fragmented to an approximate size of 35 bp by digestion with 0.1 units of DNaseI in 10 mM Tris, 2.5 mM MgCl2, 0.5 mM CaCl2, pH 7.6 for 10 minutes at 37° C. The reaction is stopped by the addition of EDTA to 5 mM final concentration. The fragments are purified with phenol extraction and ethanol precipitation. The ends of the fragments are made blunt by incubation with 1 unit of T4 DNA polymerase and 100 uM each dNTP in 50 mM NaCl, 10 mM Tris, 10 mM MgCl2, 1 mM DTT, pH 7.9 at 12° C. for 15 minutes. The reaction is stopped by the addition of EDTA to 10 mM final concentration. The fragments are purified with phenol extraction and ethanol precipitation. The ends of the fragments are dephosphorylated by incubation with 40 units of Alkaline Phosphatase in 50 mM NaCl, 10 mM Tris, 10 mM MgCl2, 1 mM DTT, pH 7.9 at 37° C. for 60 minutes. The fragments are purified with phenol extraction and ethanol precipitation. These fragments, referred to herein as target nucleic acids, are quantitated and 0.8 molar equivalents of the stem-loop adaptor oligo IA.

SEQ ID NO: 1

5′-phos-GGCCAAmCGTAmCATmCmCGmCmCTTGGmCmC3′

Here, mC indicates 5-methyl cytosine. The stem-loop adapter is ligated in a 20 uL reaction containing 1× Quick Ligation Buffer and 1 uL Quick T4 DNA ligase (New England Biolabs) at 25° C. for 5 minutes. The resulting first ligation products are purified with phenol extraction and ethanol precipitation. Simultaneous phosphorylation and nick translation reactions are performed with 10 units T4 Polynucleotide Kinase, 1 mM ATP, 1 unit of E. coli DNA Polymerase I, 33 uM each dATP, dGTP, dTTP, and 5-methyl-dCTP in 50 mM NaCl, 10 mM Tris, 10 mM MgCl2, 1 mM DTT, pH 7.9 at 25° C. for 15 minutes. The resulting strand replacement products are purified with phenol extraction and ethanol precipitation.

Oligos P1 and cP2 are pre-annealed and 1.2 molar equivalents are ligated to the strand replacement products in a 20 uL reaction containing 1× Quick Ligation Buffer and 1 uL Quick T4 DNA ligase (New England Biolabs) at 25° C. for 5 minutes. Oligo P1 and cP2 is as follows, respectively:

SEQ ID NO: 2

5′-mCmCAmCTAmCGmCmCTmCmCGmGTTTmCmCTmCTmCTATG

SEQ ID NO: 3

5′-phosCATAGAGAGGAAAGCGGAGAATGAGGAAmCmCmCGGGGmCAG

The reaction can then be immediately bisulfite converted using the MethylSEQr™ Bisulfite Conversion Kit (Applied Biosystems). The expected single nucleic acid strand is approximately 150 nt long and is ready for emulsion PCR with P1 and P2 primers, followed by SOLiD sequencing with cP1 and cIA anchor primers.

### Example 2

One microgram of genomic DNA is fragmented to an approximate size of 1 kb by shearing in a HydroShear apparatus (Genomic Solutions). The ends of the fragments are made blunt by incubation with 1 unit of T4 DNA polymerase and 100 uM each dNTP in 50 mM NaCl, 10 mM Tris, 10 mM MgCl2, 1 mM DTT, pH 7.9 at 12° C. for 15 minutes. The reaction is stopped by the addition of EDTA to 10 mM final concentration. The fragments are purified with phenol extraction and ethanol precipitation. The ends of the fragments are dephosphorylated by incubation with 10 units of Alkaline Phosphatase in 50 mM NaCl, 10 mM Tris, 10 mM MgCl2, 1 mM DTT, pH 7.9 at 37° C. for 60 minutes. The fragments are purified with phenol extraction and ethanol precipitation. Fragments are quantitated and 0.8 molar equivalents of the stem-loop adaptor oligo IA-EcoP (see below, where mC indicated 5-methyl cytosine) is ligated in a 20 uL reaction containing 1× Quick Ligation Buffer and 1 uL Quick T4 DNA ligase (New England Biolabs) at 25° C. for 5 minutes.

SEQ ID NO: 4

5′-P-CTGCTGCCAAmCGTAmCATmCmCGmCmCTTGGmCAGmCAG3′

The resulting first ligation products are purified with phenol extraction and ethanol precipitation. The first ligation product is digested with 10 units of EcoP15I (a distal-cutting restriction enzyme) in 100 mM NaCl, 50 mM Tris, 10 mM MgCl2, 1 mM DTT, 100 ug/ml BSA, 0.1 mM Sinefungin and 1 mM ATP at 37° C. for 3 hours. The 84 nt digested first ligation product is isolated by gel purification away from the larger genomic fragments. Simultaneous phosphorylation and nick translation reactions are performed with 10 units T4 Polynucleotide Kinase, 1 mM ATP, 1 unit of E. coli DNA Polymerase I, 33 uM each dATP, dGTP, dTTP, and 5-methyl-dCTP in 50 mM NaCl, 10 mM Tris, 10 mM MgCl2, 1 mM DTT, pH 7.9 at 25° C. for 15 minutes. The resulting strand replacement products are purified with phenol extraction and ethanol precipitation. Oligos P1 and cP2 are pre-annealed and 1.2 molar equivalents are ligated to the purified strand replacement products in a 20 uL reaction containing 1× Quick Ligation Buffer and 1 uL Quick T4 DNA ligase (New England Biolabs) at 25° C. for 5 minutes, to form dual-adapter ligation products. (The same oligos were used as in Example 1).

The reaction is then immediately bisulfite converted using the MethylSEQr™ Bisulfite Conversion Kit (Applied Biosystems). The expected single stranded nucleic acid is approximately 150 nt long and is ready for emulsion PCR with P1 and P2 primers, followed by for example SOLID™ sequencing with cP1 and cIA anchor primers.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method of determining the methylation profile of a target nucleic acid comprising;
ligating a first adapter to an extendable 3′ end of a 5′ dephosphorylated target nucleic acid, wherein the target nucleic acid comprises a first native strand and a complementary second strand, and wherein a nick is between a 3′ extendable end of the adapter and the second strand of the target nucleic acid;
extending the extendable 3′ end of the adapter with a strand-replacing polymerase and dATP, dGTP, dTTP, 5-methyl-dCTP, to form a fully methylated second strand, wherein the fully methylated strand is complementary to the first strand;
phosphorylating the first strand to form a phosphorylated 5′ end;
ligating the phosphorylated 5′ end of the first strand to an extendable 3′ end of a second adapter, and ligating an extendable 3′ end of the fully-methylated second strand to a phosphoryated 5′ end of the second adapter, to form a dual-adapter ligation product;
converting non-methylated cytosine in the first native strand of the dual-adapter ligation product to uracil to form a converted native strand in a converted dual-adapter ligation product; and,
comparing the identity of the cytosine positions in the fully methylated strand with the identity of the cytosine positions in the converted native strand to determine the methylation profile of the target nucleic acid.

2. The method according to claim 1 wherein the first adapter is a stem-loop adapter.

3. The method according to claim 2 wherein the stem-loop adapter comprises a 5′ phosphorylated end and an extendable 3′ end.

4. The method according to claim 1 wherein the comparing comprises performing a sequencing reaction.

5. The method according to claim 4 wherein the sequencing reaction is an enzyme-mediated extension reaction selected from the group consisting of a ligase-mediated extension of ligation probes, a polymerase-mediated extension of reversible terminators, and a polymerase mediated extension di-deoxy nucleotides.

6. The method according to claim 1 wherein the converting comprises treating with bisulfite.

7. A method of determining the methylation profile of a target nucleic acid comprising;
ligating a first adapter to an extendable 3′ end of the target nucleic acid, wherein the first adapter is a stem-loop molecule comprising an extendable 3′ end and a phosphorylated 5′ end, wherein the target nucleic acid comprises a native first strand and a complementary second strand, and wherein a nick is between the 3′ extendable end of the first adapter and the second strand of the target nucleic acid;
extending the 3′ end of the stem-loop adapter with dATP, dGTP, dTTP, 5-methyl-dCTP to form a fully methylated strand, wherein the fully methylated strand is complementary to the first native strand;
providing a second adapter, wherein the second adapter comprises a first strand and a second strand, wherein the first strand comprises a first primer portion, and an extendable 3′ end, and the second strand comprises a second primer portion and a phosphorylated 5′ end;
ligating the fully methylated second strand to the phosphorylated 5′ end of the second adapter and ligating the first native strand of the target nucleic acid to the extendable 3′ end of the second adapter, to form a dual-adapter ligation product;
converting non-methylated cytosine in the first native strand of the dual-adapter ligation product to uracil to form a converted native strand in a converted dual-adapter ligation product;
immobilizing the converted dual-adapter ligation product on a solid support;
hybridizing a primer to the second primer portion of the converted dual-adapter ligation product;
sequencing the converted dual-adapter ligation product; and,
comparing the identity of the cytosine positions in the fully-methylated second strand with the identity of the cytosine positions in the converted strand to determine the methylation profile of the target nucleic acid.

8. The method according to claim 7 wherein the sequencing reaction is an enzyme-mediated extension reaction.

9. The method according to claim 7 wherein the converting comprises treating with bisulfite.

10. The method according to claim 7 wherein the first strand of the second adapter further comprises an affinity moiety, and the immobilizing comprises interacting the affinity moiety with an affinity moiety binding partner.

11. The method according to claim 7 wherein the immobilizing comprises covalently attaching the converted dual-adapter ligation product to a bead.

12. A reaction mixture comprising;
(a) an adapter ligated to a first strand of a target nucleic acid,
wherein the target nucleic acid comprises the first strand and a second strand,
wherein the adapter is a stem-loop adapter comprising an extendable 3′ end, and,
wherein a nick exists between the extendable 3′ end of the stem-loop adapter and the second strand of the target nucleic;
(b) a strand-replacing polymerase;
(c) 5-methyl-dCTP; and,
(d) at least one of DATP, dTTP, dGTP.

13. A strand replacement product, wherein the strand replacement product comprises a high complexity fully methylated strand and a low complexity converted native strand.

14. The composition according to claim 13 wherein the high complexity fully methylated strand comprises 5-methyl-dCTP.

15. A kit for determining the methylation profile of a target nucleic acid comprising;
(a) a first adapter, wherein the first adapter is a stem-loop adapter, and wherein the stem-loop adapter comprises a phosphorylated 5′ end and an extendable 3′ end;
(b) a second adapter, wherein the second adapter comprises a phosphorylated 5′ end;
(c) a strand-replacing polymerase;
(d) a converting agent;
(e) a kinase;
(f) 5-methyl-dCTP; and,
(g) at least one of dATP, dTTP, dGTP.

16. The kit according to claim 15 further comprising;
(h) a distal-cutting restriction enzyme.

17. The kit according according to claim 15 further comprising;
(i) sequencing reagents.

18. The kit according to claim 15 wherein the converting agent is bisulfite.

19. A method of mapping a low complexity sequence to a locus of a genome comprising;
generating a strand replacement product comprising a high complexity strand and a low complexity strand;
sequencing the high complexity strand; and,
comparing the sequence of the high complexity strand to the genome in order to map the low complexity strand to a locus of the genome.

20. The method according to claim 19 wherein the high complexity strand is a fully methylated strand and the low complexity strand is a converted native strand.

21. The method according to claim 19 wherein the fully-methylated strand comprises cytosines that are methylated, and the strand-replacing reaction comprises 5-methyl-dCTP.

22. The method according to claim 19 wherein the fully methylated strand comprises adenines that are methylated, and the strand replacing reaction comprises methylated adenines.

23. A method of forming a single-stranded dual-adapter ligation product comprising;
forming an adapter-ligated single-stranded target nucleic acid;
hybridizing a primer to the adapter of the adapter-ligated single-stranded target nucleic acid;
extending the primer in the presence of 5-methyl dCTP to form a double-stranded product comprising a fully methylated strand; and,
ligating a stem-loop adapter to the double-stranded product to form a single-stranded dual adapter ligation product.

24. The method according to claim 23 wherein the single-stranded dual adapter ligation product is treated with a converting reagent and sequenced to determine the methylation status of a target nucleic acid.


SEQUENCE LISTING (4 sequences)

SEQ ID NO: 1 - DNA, 24 bp, Artificial
  Synthetic
ggccaacgtacatccgccttggcc

SEQ ID NO: 2 - DNA, 28 bp, Artificial
  Synthetic
ccactacgcctccgctttcctctctatg

SEQ ID NO: 3 - DNA, 38 bp, Artificial
  Synthetic
catagagaggaaagcggagaatgaggaacccggggcag

SEQ ID NO: 4 - DNA, 32 bp, Artificial
  Synthetic
ctgctgccaacgtacatccgccttggcagcag